(19) United States
(12) Patent Application Publication
(10) Pub. No.: US 2011/0189113 A1
(43) Pub. Date: Aug. 4, 2011

(54) Water-Resistant, Rub-Resistant, Sprayable Homogeneous Sunscreen Composition

(76) Inventors: Tracey Ross, Hewitt, NJ (US); Christine Barrett, Oakland, NJ (US); Nancy Clements, North Plainfield, NJ (US); Sangeetha Subramanian, Pomona, NY (US)

(21) Appl. No.: 12/673,316

(22) PCT Filed: Aug. 12, 2008
(86) PCT No.: PCT/US08/72883

§ 371 Date: Jan. 31, 2011

(51) Int. Cl. A61K 8/81; A61Q 17/04

(52) U.S. Cl. 424/59

(57) ABSTRACT

In accordance with the present invention, there is provided a water-resistant, rub-resistant, sprayable, clear, and homogeneous sunscreen composition which includes an active sunscreen ingredient, alcohol, and a vinyl lactam or maleimide polymer soluble in the composition.

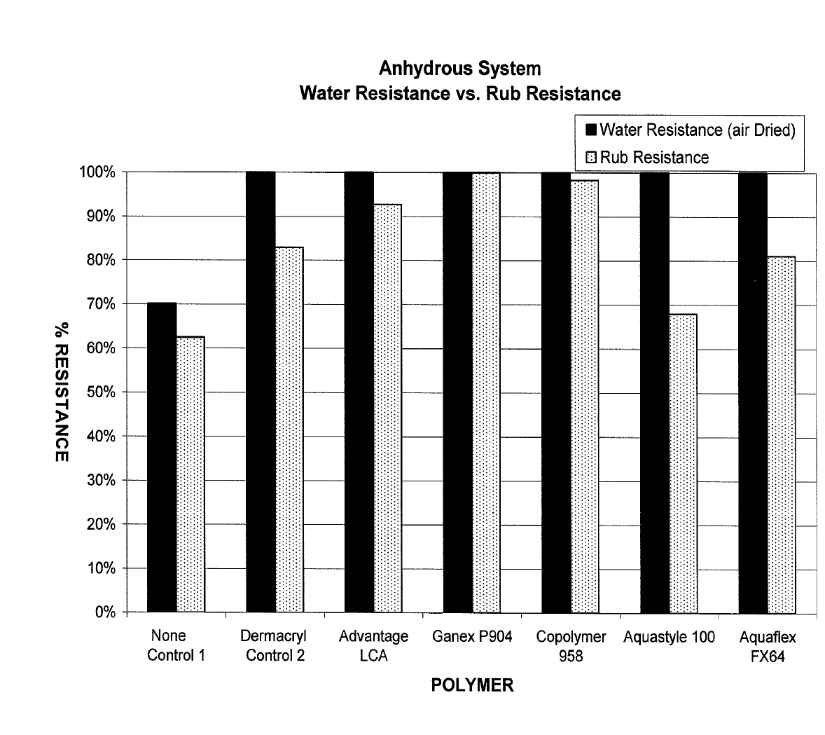

### CROSS REFERENCE TO RELATED APPLICATION

This Application claims the benefit of U.S. Provisional Application Ser. No. 60/955,499 filed Aug. 13, 2007, the contents of which are hereby incorporated by reference.

### FIELD OF THE INVENTION

This invention relates to sunscreen compositions, and more particularly, to anhydrous, water-resistant, rub-resistant, sprayable, clear, homogeneous sunscreen compositions containing a vinyl lactam or maleimide polymer soluble in the composition as an efficacious ingredient to impart these desirable properties to the composition.

### BACKGROUND OF THE INVENTION

The sunscreen market has recently turned to alcohol-based spray compositions containing acrylate-based polymers to provide water-proofing properties for the user. See H. Caoletal, U.S. Publication No. US2005/00112080, published May 26, 2005.

Enhanced SPF UV/sunscreen/tricontanyl PVP photoprotecting (sprayable) formulations are described in U.S. Pat. No. 6,436,376. However, such tricontanyl PVP polymer (e.g., Ganex WP-660) containing compositions do not provide anhydrous, homogeneous compositions.

Accordingly, it is an object of the present invention to provide new and improved anhydrous sunscreen compositions which can provide both excellent water-resistance and rub-resistance properties which also find acceptability by the consumer.

Another object herein is to provide anhydrous sunscreen compositions containing a soluble polymer ingredient which imparts these properties in a sprayable, clear and homogeneous composition for the user.

### SUMMARY OF THE INVENTION

What has been discovered herein is a water-resistant, rub-resistant, sprayable, clear sunscreen composition which includes (a) an active sunscreen ingredient, (b) alcohol and (c) a vinyl lactam or maleimide polymer.

Suitably, the composition is an anhydrous or hydroalcoholic composition, preferably anhydrous. Preferably, (c) is polyvinyl pyrrolidone or vinyl caprolactam, e.g. butylated polyvinyl pyrrolidone; vinyl caprolactam/vinyl pyrrolidone/dimethylaminoethyl methacrylate; or a maleimide copolymer (Advantage LCA). A most preferred composition includes by wt, (a) 5-40%; (b) 5-88%; and (c) 0.25-10%, most preferably (a) is 15-35%; (b) 50-80%; and (c) 0.5-10%.

### BRIEF DESCRIPTION OF THE FIGURE

The FIG. is a bar graph of water-resistance and rub-resistance for particular compositions in accordance with certain aspects of the invention vs. control formulations.

### DETAILED DESCRIPTION OF THE INVENTION

The sunscreen compositions of the invention can contain one or more active UVA and UVB compounds, e.g., avobenzone, benzophenone-3, p-Aminobenzoic acid (PABA), Camphor benzalkonium methosulfate, Phenylbenzimidazole sulfonic acid, Terephthalidene dicamphor sulfonic acid, Benzylidene camphor sulfonic acid, Octocrylene, Polyacrylamidomethyl benzylidene camphor, Ethylhexyl methoxycinnamate, PEG-25 PABA, Isoamyl p-methoxycinnamate, Ethylhexyl triazone, Drometrizole trisiloxane, Diethylhexyl butamido triazone, 4-Methylbenzylidene camphor, 3-Benzylidene camphor, Ethylhexyl salicylate, Ethylhexyl dimethyl PABA, Benzophenone-4, Benzophenone-5, Methylene bis-benztriazolyl tetramethylbutylphenol, Disodium phenyl dibenzimidazole tetrasulfonate, Bis-ethylhexyloxyphenol methoxyphenol triazine, and Polysilicone-15. Other compounds described in the art for the purpose may also be used.

Water resistance as set forth herein is measured in accordance with the following procedure. In-vitro skin (IMS Testing Group) is hydrated for 12 to 18 hours in a hydration chamber (82% water/18% glycerin solution). Four (4) samples and one reference/blank are prepared by applying 6 to 8 mg. of the sunscreen sample onto each sample and allowing the sample to re-hydrate for 20 minutes.

Initial UV absorbance readings are measured (each sample vs. the reference) for each sample scanned at four orientations rotating 90 degrees after each reading. Readings are taken at the highest peak between 250-400 nm on a dual beam UV-Vis spectrophotometer with slide holder. The four (4) samples are placed in the water bath (25° C. with propeller mixing at 50 rpm) for 80 minutes. The samples are removed from the water bath and allowed to dry for 10 minutes. The samples are placed in the hydration chamber for 120 minutes. Readings are taken (each sample vs. the reference) for each sample scanned at four orientations rotating 90 degrees after each reading. Readings are taken at the highest peak between 250-400 nm.

Retention (water resistance) is calculated by dividing the final readings by the initial readings and multiplying by 100. The average and standard deviation are calculated and recorded. Inconsistently high or low values should be omitted from the calculation of the retention.

Compositions according to certain aspects of the present invention provide water resistance (as determined by the procedures set forth herein) of at least 90%, more particularly at least 95%, and in certain cases about 100%. The described in vitro test method has been confirmed through corresponding in vivo tests.

Rub resistance as set forth herein is measured in accordance with the following procedure which simulates swimming and towel drying. In-vitro skin (IMS Testing Group) is hydrated for 12 to 18 hours in a hydration chamber (82% water/18% glycerin solution). Four (4) samples and one reference/blank are prepared by applying 6 to 8 mg. of the sunscreen sample onto each sample and allowing the sample to re-hydrate for 20 minutes.

Initial UV absorbance readings are taken (each sample vs. the reference) for each sample scanned at four orientations rotating 90 degrees after each reading. Readings are taken at the highest peak between 250-400 nm using a UV-Vis spectrophotometer with slide holder (two beam).

The completed samples are placed in the water bath (25° C. with propeller mixing) for 80 minutes. The samples are removed from water bath. Double-sided tape is placed on the underside of the sample slides and placed on a Friction machine (Gardco: Washability and Wear Tester—Linear Motion Test Equipment Model D10VF/Cat#WA-2155), sample side up.

After the samples are secured, a cotton towel (4″×5″ piece) is wrapped around the friction boat (abrasion boat with auxiliary 1 lb. weight measuring 2″×4″) and secured with double-sided tape. The boat is placed into the inner side of the arm of the Wear Tester. The Wear tester is set at Speed 3 and the towel is allowed to make 3.5 passes (one pass is to the left and then back to the right to the starting position). After the wear test is completed, the samples and tape are removed and the samples are placed in the hydration chamber for 120 minutes.

Final readings are taken (each sample vs. the reference) for each sample scanned at four orientations rotating 90 degrees after each reading. The readings are taken at the highest peak between 250-400 nm. Retention (rub resistance) is calculated by dividing the final readings by the initial readings and multiplying by 100. The average and standard deviation are calculated and recorded. Inconsistently high or low values should be omitted from the calculation of the retention.

Compositions according to certain aspects of the present invention provide rub resistance (as determined by the process described herein) of at least 75%, more particularly at least 85%, still more particularly at least 95%, and in certain cases about 100%.

The invention will now be described by reference to the following non-limiting examples of invention compositions.

### Example 1

| | | Wt % | Source |
|---|---|---|---|
| (a) | Active Sunscreen Ingredient | | |
| | Homosolate | 5.5 | NeoHeliopan |
| | | | (Symrise) |
| | Octinoxate | 7.5 | Escalol 557 |
| | | | (ISP) |
| | Octisalate | 5.0 | Escalol 587 |
| | | | (ISP) |
| | Oxybenzone | 4.0 | Escalol 567 |
| | | | (ISP) |
| (b) | Alcohol | | |
| | SD 40 Ethanol | 75.3 | |
| (c) | Vinyl Lactam Copolymer | | |
| | Vinyl caprolactam/vinyl pyrrolidone/ | 2.7 | Advantage LCA |
| | Dimethylaminoethyl Methacrylate | (1% Solid) | (ISP) |
| | Copolymer (37% in Ethanol) | | |
| | | 100.0 | |

1. The composition in example 1 (SFP-25) was a clear, straw-colored, sprayable water-thin liquid formulation.

2. The water-resistance was measured at 100%.

3. The rub-resistance was measured at 92.7%.

### Example 2

| | | Wt % | Source |
|---|---|---|---|
| (a) | Active Sunscreen Ingredient | | |
| | Homosalate | 5.5 | |
| | Octinoxate | 7.5 | |
| | Octisalate | 5.0 | |
| | Oxybenzone | 4.0 | |
| (b) | Alcohol | | |
| | SD 40 | 77.0 | |
| (c) | Vinvl Lactam Polymer | | |
| | Butylated Vinyl Pyrrolidone | 1.0 | GANEX ® P904 |
| | | (1% solid) | (ISP) |
| | | 100.0 | |

1. The composition in Example 2 was a clear, straw-colored, sprayable water-thin liquid formulation.

2. The water resistance was measured at 100%.

3. The rub-resistance also was measured at 100%.

### Example 3

| | | Wt % | Source |
|---|---|---|---|
| (a) | Active Sunscreen Ingredient | | |
| | Homosolate | 5.5 | NeoHeliopan |
| | | | (Symrise) |
| | Octinoxate | 7.5 | Escalol 557 |
| | | | (ISP) |
| | Octisalate | 5.0 | Escalol 587 |
| | | | (ISP) |
| | Oxybenzone | 4.0 | Escalol 567 |
| | | | (ISP) |
| (b) | Alcohol | | |
| | SD 40 Ethanol | 75.5 | |
| (c) | Maleimide Copolymer | | |
| | Isobutylene/ethylmaleimide/ | 2.5 | Aquaflex FX-64 |
| | hydroxyethyl maleimide | (1% solid) | (ISP) |
| | | 100.0 | |

1. The composition in Example 3 was a clear, straw-colored, sprayable water-thin liquid formulation.

2. The water resistance was measured at 100%.

3. The rub resistance was measured at 81%.

The compositions described in Examples 1 and 2 were also prepared using the following vinyl lactam copolymers (at 1% polymer):

PVP/dimethylaminoethyl methacrylate (Copolymer No. 958 (ISP)-50% in ethanol) (water resistance—100%; rub resistance—98%); vinyl pyrrolidone/acrylates/lauryl methacrylate (STYLEZE® 2000-ISP); and vinyl pyrrolidone/methaminopropyl aminopropyl methacrylamide (AQUASTYLE®-ISP) (water resistance—100%; rub resistance—68%).

These compositions also were anhydrous spray formulations with excellent water-resistance and rub-resistance.

A composition with an SPF of 50 was also prepared using Copolymer No. 958 (ISP). The SPF 50 anhydrous system provided a water resistance of 98% and a rub resistance of 86%.

CONTROL 1

| | | Wt % |
|---|---|---|
| (a) | Active Sunscreen Ingredient | |
| | Homosolate | 5.5 |
| | Octinoxate | 7.5 |
| | Octisalate | 4.0 |
| | Oxybenzone | 5.5 |
| (b) | Alcohol | |
| | SD40 | 78.0 |
| (c) | Polymer | |
| | None | |

1. The water-resistance was measured at 70.0%.

2. The rub-resistance was measured at 62.5%

CONTROL 2

| | | Wt % | Source |
|---|---|---|---|
| (a) | Active Sunscreen Ingredient | | |
| | Homosalate | 5.5 | |
| | Octimoxate | 7.5 | |
| | Octisalate | 5.0 | |
| | Oxybenzone | 4.0 | |
| (b) | Alcohol | | |
| | SD40 | 77.0 | |
| (c) | Polymer | | |
| | DERMACRYL ® 79 | 1.0 | National Starch |

1. Armeen DM 18D was used as a neutralizer in an amount of 0.05.

2. The anhydrous benchmark control spray formulation had a water-resistance of 100%.

3. The rub-resistance of the formulation was measured at 87.6%.

### Control 3

PVP-eicosene (GANEX® 220) copolymer was used in place of Ganex P904 in the sunscreen formulation of Example 2. A non-homogeneous composition resulted because the copolymer was not soluble in the formulation.

While vinyl lactam polymers are particularly effective solubilized in the anhydrous systems described herein, maleimide copolymers, such as the maleimide copolymer sold as Aquaflex FX-64 by ISP perform similarly as well. Such copolymers include copolymers of isobutylene/ethylmaleimide/hydroxyethyl maleimide monomers. These polymers are described by Ulmer et al in U.S. Pat. No. 6,025,501, the disclosure of which is hereby incorporated by reference in its entirety.

Moreover, it has been found that the sunscreen compositions of this invention exhibit outstanding aesthetics, e.g., a desirable skin feel, and are not sticky or tacky.

The invention has been described in detail with particular reference to preferred embodiments thereof, but it will be understood that variations and modifications can be effected within the spirit and scope of the invention.

What is claimed:

1. A water-resistant, rub-resistant, sprayable, clear, homogeneous sunscreen composition comprising:
(a) an active sunscreen ingredient;
(b) alcohol; and
(c) a vinyl lactam or maleimide polymer soluble in said composition.

2. The composition of claim 1 which is an anhydrous composition.

3. The composition of claim 1 wherein (c) is vinyl pyrrolidone.

4. The composition of claim 1 wherein (c) is butylated polyvinyl pyrrolidone.

5. The composition of claim 1 wherein (c) is vinyl caprolactam.

6. The composition of claim 1 wherein (c) is a vinylcaprolactam/vinyl pyrrolidone/dimethylaminoethyl methacrylate copolymer.

7. The composition of claim 1 wherein (b) is ethanol.

8. The composition of claim 1 wherein by wt %; (a) is 5-40%; (b) is 50-88%; and (c) is 0.25-10%.

9. The composition of claim 8 wherein (a) is 15-35%; (b) is 50-80%; and (c) is 0.5-10%.

10. The composition of claim 8 wherein (a) is about 22%; (b) is about 77%; and (c) is about 1%.

11. The composition of claim 1 wherein (c) is a maleimide polymer.

12. The composition of claim 11 wherein said maleimide polymer is derived from isobutylene/ethylmaleimide/hydroxyethyl maleimide monomers.

13. The composition of claim 1 wherein said composition provides a water resistance of at least 90%.

14. The composition of claim 13 wherein said composition provides a water resistance of at least 95%.

15. The composition of claim 1 wherein said composition provides a rub resistance of at least 75%.

16. The composition of claim 15 wherein said composition provides a rub resistance of at least 85%.

17. The composition of claim 16 wherein said composition provides a water resistance of about 100%.
